(19) United States
(12) Patent Application Publication
(10) Pub. No.: US 2015/0153340 A1
(43) Pub. Date: Jun. 4, 2015

(54) COMPOSITION FOR DIAGNOSIS OF BEHCET'S DISEASE

(71) Applicant: AJOU UNIVERSITY INDUSTRY-ACADEMIC COOPERATION FOUNDATION, Suwon-si (KR)

(72) Inventor: Seong Hyang SOHN, Suwon-si (KR)

(21) Appl. No.: 14/310,851

(22) Filed: Jun. 20, 2014

(30) Foreign Application Priority Data
Nov. 29, 2013 (KR) 10-2013-0147599

(51) Int. Cl. G01N 33/569

(52) U.S. Cl. CPC G01N 33/56966; G01N 2333/70596

(57) ABSTRACT

The present invention relates to a composition for the diagnosis of Behcet's disease, comprising an agent for measuring the expression level of macrophage mannose receptor 1 (CD206), a kit for the diagnosis of Behcet's disease, and an information providing method using the same. According to the present invention, it is possible to diagnose the pathogenesis of Behcet's disease without being affected by symptoms of Behcet's disease or prescription drugs and differentially diagnose between active Behcet's disease and inactive Behcet's disease.

This application claims the benefit of Korean Patent Application No. 10-2013-0147599, filed on Nov. 29, 2013, in the Korean Intellectual Property Office, the disclosure of which is incorporated herein in its entirety by reference.

### FIELD

The present invention relates to a composition for the diagnosis of Behcet's disease, a kit for the diagnosis of Behcet's disease, and a method for providing information for the diagnosis of Behcet's disease.

### BACKGROUND

Behcet's disease is a rare chronic inflammatory disease characterized by recurrent oral and/or genital aphthous ulcerations, uveitis and skin lesions. Clinical presentation of this disorder is multifaceted with severe chronic inflammation accompanied by articular, central nervous system, gastrointestinal, renal, urogenital, pulmonary and cardiovascular manifestations, all of which are associated with systemic vasculitis, a pivotal pathophysiological feature of Behcet's disease. The exact pathogenesis of Behcet's disease remains unclear, but autoimmune and autoinflammatory reactions are important.

Initially, in Behcet's disease, infiltrated types of cells include CD4+ and CD8+ T cells, macrophages and dendritic cells, followed by neutrophils. Th1/Th2-type immune responses have been investigated in cell-mediated immunity and inflammation in Behcet's disease. T helper (Th) 1 and Th17 predominant response has been observed in many studies in patients with Behcet's disease; the response involves the increased production of cytokines including interleukin (IL)-2, IL-6, IL-8, IL-17, IL-12, IL-18, tumor necrosis factor-alpha (TNF-α), and interferon-gamma (IFN-gamma).

CD11b is expressed on neutrophils, monocytes, natural killer (NK) cells and a subset of lymphocytes. CD11b has been implicated as having a central role in the migration of leukocytes from peripheral blood to the sites of inflammation, and is also involved in adhesion, chemotaxis and diapedesis during the process of host defense. A previous study reported the significantly elevated expression of the CD11a, CD11b and CD18 adhesion molecules compared to cells from healthy subjects.

CD14, the receptor for lipopolysaccharide binding protein, is expressed to a higher degree in blood monocytes than in tissue macrophages and is a co-receptor of innate immunity. Behcet's disease patients display up-regulated CD14 expression on monocytes and neutrophils and elevated serum soluble CD14 levels. The activation confirmed by CD69 and CD14 response to heat shock protein 60 (HSP60) on peripheral blood mononuclear cells (PBMCs) of Behcet's disease patients might be associated with an innate activation induced through antigen presenting cells (APCs).

CD16 is an Fc receptor (Fc RIII) that has been directly associated with neutrophil activation. Normally, CD14 and CD16 are found together in secretory vesicles of neutrophils and, when neutrophils are stimulated, CD14 and CD16 comigrate to the plasma membrane together. The intensity of CD16 expression in patients with Behcet's disease is equivocal. CD16⁺(FcγIIIA) is expressed on NK cells, macrophages, and neutrophils. FcγRII, namely CD32, has been detected on T cells, mast cells, monocytes, macrophages, and some epithelial and endothelial cell lineages. CD32 is important in regulating adaptive immunity, and the primary function of CD32 appears to be antibody-mediated uptake of antigen and modulation of cellular activation and maturation events.

CD206, the macrophage mannose receptor (MMR), is a scavenger receptor that is expressed primarily by tissue macrophages and lymphatic and hepatic endothelia. MMR's carbohydrate pattern recognition and role in phagocytosis of microorganisms support a dual role in host defense and homeostasis. In addition, CD206 has been identified in a variety of autoimmune and inflammatory diseases, such as systemic lupus erythematosus, ulcerative colitis, and Crohn's disease. However, the role of mannose receptor on PBMCs of active and inactive Behcet's disease patients remain poorly understood in the host defense.

It has been reported that CD8⁺CD11⁺ is highly expressed in Behcet's disease patients, but the results of the expression are different depending on symptoms of Behcet's disease or the types of drugs prescribed for the treatment, which makes it impossible to use it as a marker for the diagnosis of Behcet's disease and to differentially diagnose between active Behcet's disease and inactive Behcet's disease, and thus research and development of biomarkers that can differentially diagnose between active Behcet's disease and inactive Behcet's disease has been continuously required.

Relevant Patent Document: Korean Patent No.: 10-0448488

### SUMMARY

The present application has been made in an effort to solve the above-described problems associated with prior art, and an object of the present application is to provide a composition for the diagnosis of Behcet's disease, which uses macrophage mannose receptor 1 as a bio-marker that can diagnose Behcet's disease, particularly differentially diagnose between active Behcet's disease and inactive Behcet's disease.

To this end, the present inventors have investigated the pattern of cell-surface expression of CD11b, CD14, CD16, CD32 and CD206 on PBMCs of Behcet's disease patients for the diagnosis of active Behcet's disease and finally invented a composition that can use CD206 as a marker for the diagnosis of active Behcet's disease.

To achieve the above object, the present application provides a composition for the diagnosis of Behcet's disease, comprising an agent for measuring the expression level of macrophage mannose receptor 1 (CD206).

Moreover, the present application provides a kit for the diagnosis of Behcet's disease, comprising an agent for measuring the expression level of macrophage mannose receptor 1 (CD206).

Furthermore, the present application provide a method for providing information for the diagnosis of Behcet's disease, comprising the steps of providing a sample to be analyzed, contacting an antibody specific to macrophage mannose receptor 1 (CD206) with the sample, and measuring the expression level of CD206.

### BRIEF DESCRIPTION OF THE DRAWINGS

The above and other features and advantages of the present invention will become more apparent by describing in detail exemplary embodiments thereof with reference to the attached drawings in which:

FIGS. 1A to 1E are graphs showing the frequencies of expression of proteins on the surface of PBMCs, in which FIGS. 1A to 1E show the surface expression of CD11⁺, CD14⁺, CD16⁺, CD32⁺, and CD206⁺, respectively, the horizontal axis of the graph represents each experimental group where HC represents a healthy control group (6 subjects), Inactive represents a patient group with inactive Behcet's disease (8 subjects), and Active represents a patient group with active Behcet's disease (5 subjects), and the vertical axis of the graph represents the expression degree (%);

FIGS. 2A to 2C are graphs showing the frequencies of expression of proteins on the surface of PBMCs, in which FIGS. 2A to 2C show the surface expression of CD11b⁺CD14⁺, CD11b⁺CD16⁺, and CD11b⁺CD32⁺, respectively, the horizontal axis of the graph represents each experimental group where HC represents a healthy control group (6 subjects), Inactive represents a patient group with inactive Behcet's disease (8 subjects), and Active represents a patient group with active Behcet's disease (5 subjects), and the vertical axis of the graph represents the expression degree (%);

FIGS. 3A and 3B are graphs showing the frequencies of expression of proteins on the surface of PBMCs, in which FIGS. 3A and 3B show the surface expression of CD14⁺CD16⁺ and CD14⁺CD32⁺, respectively, the horizontal axis of the graph represents each experimental group where HC represents a healthy control group (6 subjects), Inactive represents a patient group with inactive Behcet's disease (8 subjects), and Active represents a patient group with active Behcet's disease (5 subjects), and the vertical axis of the graph represents the expression degree (%);

FIGS. 4A and 4B are graphs showing the frequencies of expression of proteins on the surface of PBMCs, in which FIGS. 4A and 4B show the surface expression of CD11⁺CD206⁺ and CD14⁺CD206⁺, respectively, the horizontal axis of the graph represents each experimental group where HC represents a healthy control group (6 subjects), Inactive represents a patient group with inactive Behcet's disease (8 subjects), and Active represents a patient group with active Behcet's disease (5 subjects), and the vertical axis of the graph represents the expression degree (%);

FIGS. 5A to 5C are figures showing the frequencies of expression of proteins on the surface of PBMCs in patients improved from active to inactive;

FIG. 6 is a graph showing the IL-10 serum levels in Behcet's disease patients, in which the horizontal axis of the graph represents the experimental group where Active BD represents a patient with active Behcet's disease and Inactive BD represents a patient with inactive Behcet's disease and the vertical axis of the graph represents the IL-10 level (pg/ml); and

FIG. 7 shows transmission electron microscopy images of PBMCs, in which Active represents the PBMCs in a patient group with active Behcet's disease, Inactive represents the PBMCs in a patient group with inactive Behcet's disease, and Healthy represents the PBMCs in a healthy control group.

### DETAILED DESCRIPTION

As used herein, unless otherwise specified, the term “specific” or “specifically” refers to the ability of binding to a target protein without affecting other proteins in cells and refers to the specificity to macrophage mannose receptor 1 in the present invention.

As used herein, unless otherwise specified, the term “diagnosis” refers to the determination of the presence or characteristics of a pathological condition, and for the purpose of the present invention, it refers to the diagnosis of the pathogenesis of Behcet's disease, most preferably, the determination of the pathogenesis of active Behcet's disease.

As used herein, unless otherwise specified, the term “sample to be analyzed” refers to a biological sample such as a subject's blood or peripheral blood mononuclear cells (PBMCs) isolated from the blood, from which the differential expression of marker proteins can be detected by the pathogenesis of Behcet's disease, and may be treated and prepared by a method commonly used in the art of the present invention.

As used herein, unless otherwise specified, the term “diagnostic marker”, “marker for the diagnosis”, or “diagnosis marker” refers to a material capable of determining the expression level of macrophage mannose receptor 1 and preferably refers to organic biomolecules that are differentially expressed on the surface of peripheral blood mononuclear cells between normal subjects and active Behcet's disease subjects.

As used herein, unless otherwise specified, the term “antigen-antibody complex” refers to a complex of macrophage mannose receptor 1(CD206) in a biological sample and an agent specifically recognizing macrophage mannose receptor 1. Experimental methods for investigating the formation of antigen-antibody complexes include immunohistochemical staining, radioimmunoassay (RIA), enzyme-linked immunosorbent assay (ELISA), Western Blotting, immunoprecipitation assay, complement fixation assay, flow cytometry, protein chip, etc.

As used herein, unless otherwise specified, the term “antibody” refers to a specific protein molecule that indicates an antigenic region. For the purpose of the present invention, the antibody specifically binds to macrophage mannose receptor 1 and includes polyclonal antibodies, monoclonal antibodies, recombinant antibodies, and combinations thereof.

Hereinafter, the present invention will be described in more detail.

The composition for the diagnosis of Behcet's disease of the present application comprises an agent for measuring the expression level of macrophage mannose receptor 1 (CD206).

Behcet's disease is a rare chronic inflammatory disease and is divided into active Behcet's disease with symptoms and inactive Behcet's disease without symptoms. There is currently no definitive diagnostic method for the diagnosis of Behcet's disease, and the diagnosis of Behcet's disease is performed by skin hyperreactivity (pathergy test), genetic testing (HLA-B₅₁ detection), erythrocyte sedimentation rate (ESR), and C-reactive protein (CRP) level, but these tests are not definitive methods, but are common inflammatory tests. Via the pathergy test and genetic testing, it is difficult to differentially diagnose between active Behcet's disease patients and inactive Behcet's disease patients. Behcet's disease to be diagnosed in the present invention may preferably be active Behcet's disease.

Macrophage mannose receptor 1 (CD206) is a protein encoded by the MRC1 gene and is also known as C-type mannose receptor 1 (MMR1). Macrophage mannose receptor 1 (CD206) is a C-type lectin carbohydrate-binding protein, which is primarily present on the surface of macrophages and dendritic cells, and is found in human skin fibroblasts and keratinocytes.

The present inventors determined that the frequency of peripheral blood mononuclear cells representing the CD206 surface marker specifically increased in active Behcet's disease patients, indicating that it is possible to diagnose Behcet's disease using the composition comprising an agent for measuring the expression level of CD206 and, in particular, to differentially diagnose between active Behcet's disease and inactive Behcet's disease, and thus the CD206 protein can be used as a marker for the diagnosis of Behcet's disease.

The agent for measuring the expression level of CD206 may preferably be an antibody specific to macrophage mannose receptor 1 and more preferably an antibody to CD206 expressed on the surface of peripheral blood mononuclear cells. Examples of the agent for measuring the expression level of CD206 include those capable of measuring the single expression of CD206 as well as those capable of measuring the co-expression of CD206CD11b or CD206CD14.

The antibody means a specific protein molecule that indicates an antigenic region and, for the purpose of the present invention, means an antibody specifically recognizing CD206.

Examples of the antibody include polyclonal antibodies, monoclonal antibodies, recombinant antibodies, and complete forms having two full-length light chains and two full-length heavy chains as well as functional fragments of antibody molecules such as Fab, F(ab′), F(ab′)2 and Fv.

Since the CD206 has been already identified, the production of antibodies using the same can be easily performed by techniques well known in the art to which the present invention pertains, and commercially available antibodies can also be used.

The polyclonal antibodies can be produced by a method of injecting the CD206 protein antigen into an animal, collecting blood from the animal and obtaining serum containing antibodies from the collected blood. These polyclonal antibodies can be produced from any animal species host such as goats, rabbits, sheep, monkeys, horses, pigs, cattle, dogs, etc.

The monoclonal antibodies can be produced by fusion method known in the art to which the present invention pertains (Kohler and Milstein (1976) European Journal of Immunology 6:511-519), recombinant DNA method, or phage antibody library technology (Clackson et al., Nature, 352, 624-628, 1991: Marks et al., J. Mol. Biol., 222,581-597, 1991).

The antibodies may be those commercial available and, in an embodiment of the present invention, the PE-Cy™ mouse-anti-human CD206 was used.

The composition may further include any one selected from the group consisting of an agent capable of measuring the co-expression of CD206CD11b, an agent capable of measuring the co-expression of CD206CD14, and a combination thereof. The co-expression of CD206CD11 and the co-expression of CD206CD14 refer to the expression of CD11b or CD14 together with CD206 on the surface of peripheral blood mononuclear cells. The measurement of the co-expression may be performed using an agent for measuring the expression level of CD11b and/or an agent for measuring the expression level of CD14 together with the agent for measuring the expression level of CD206.

The composition may further include a label enabling quantitative or qualitative measurement of the formation of antigen-antibody complexes, and tools, reagents, etc., which are commonly used for immunological analysis, in addition to the agent for measuring the expression level of CD206.

Examples of the label enabling quantitative or qualitative measurement of the formation of antigen-antibody complexes may include enzymes, fluorescent substances, ligands, luminescent substances, microparticles, redox molecules, radioactive isotopes, etc., but not limited thereto. Examples of the enzymes available as detection labels include β-glucuronidase, β-D-glucosidase, β-D-galactosidase, urease, peroxidase, alkaline phosphatase, acetylcholinesterase, glucose oxidase, hexokinase and GDPase, RNase, glucose oxidase and luciferase, phosphofructokinase, phosphoenolpyruvate carboxylase, aspartate aminotransferase, phosphenolpyruvate decarboxylase, β-latamase, etc., but not limited thereto. Examples of the fluorescent substances include fluorescein, isothiocyanate, rhodamine, phycoerythrin, phycocyanin, allophycocyanin, o-phthalaldehyde, fluorescamine, etc., but are not limited thereto. Examples of the ligands include biotin derivatives, etc., but are not limited thereto. Examples of the luminescent substances include acridinium esters, luciferin, luciferase, etc., but are not limited thereto. Examples of the microparticles include colloidal gold, colored latex, etc., but are not limited thereto. Examples of the redox molecules include ferrocene, ruthenium complexes, viologen, quinone, Ti ions, Cs ions, diimide, 1,4-benzoquinone, hydroquinone, K₄ W(CN)⁸, [Os(bpy)₃]²⁺, [RU(bpy)₃]²⁺, [MO(CN)₈]⁴⁻, etc., but are not limited thereto. Examples of the radioactive isotopes include 3H, 14C, 32P, 35S, 36Cl, ⁵¹Cr, ⁵⁷Co, ⁵⁸Co, ⁵⁹Fe, ⁹⁰Y, ¹²⁵I, ¹³1I, ¹⁸⁶Re, etc., but are not limited thereto.

Examples of the tools or reagents include suitable carriers, solubilizing agents, detergents, buffering agents, stabilizing agents, etc., but are not limited thereto. When the labeling substance is an enzyme, the composition may include a substrate allowing the measurement of enzyme activity and a reaction terminator. Suitable carriers include soluble carriers and insoluble carriers. Examples of the soluble carriers include physiologically acceptable buffers known in the art, for example, PBS, and examples of the insoluble carriers include polystyrene, polyethylene, polypropylene, polyesters, polyacrylonitrile, fluorocarbon resin, crosslinked dextran, polysaccharides, and other papers, glasses, metals, agarose, and combinations thereof.

When the composition is used, it is possible to diagnose Behcet's disease and, in particular, to differentially diagnose between active Behcet's disease and inactive Behcet's disease. Moreover, when the CD206 is used in the diagnosis, the results of the diagnosis will not vary depending on symptoms of the disease such as whether it is accompanied by ocular lesions, and thus it is possible to reliably and accurately diagnose the disease and to diagnose the pathogenesis of active Behcet's disease regardless of the types of prescription medicines.

In another aspect of the present invention, the kit for the diagnosis of Behcet's disease comprises an agent for measuring the expression level of macrophage mannose receptor 1 (CD206).

Examples of the agent for measuring the expression level of CD206 include those capable of measuring the single expression of CD206 as well as those capable of measuring the co-expression of CD206CD11b or CD206CD14.

The co-expression of CD206CD11 and the co-expression of CD206CD14 preferably refer to the expression CD11b or CD14 together with CD206 on the surface of peripheral blood mononuclear cells. The measurement of the co-expression may be performed using an agent for measuring the expression level of CD11b and/or an agent for measuring the expression level of CD14 together with the agent for measuring the expression level of CD206.

The kit may include tools, reagents, etc., which are commonly used for immunological analysis in the art, in addition to the agent for measuring the expression level of CD206.

Examples of the tools or reagents include suitable carriers, labeling substances capable of generating detectable signals, chromophores, solubilizing agents, detergents, buffering agents, stabilizing agents, etc., but are not limited thereto. When the labeling substance is an enzyme, the kit may include a substrate allowing the measurement of enzyme activity and a reaction terminator. Suitable carriers include soluble carriers and insoluble carriers. Examples of the soluble carriers include physiologically acceptable buffers known in the art, for example, PBS, and examples of the insoluble carriers include polymers such as polystyrene, polyethylene, polypropylene, polyesters, polyacrylonitrile, fluorocarbon resin, crosslinked dextran, polysaccharides, magnetic microparticles composed of latex plated with metals, and other papers, glasses, metals, agarose, and combinations thereof.

When the kit is used, it is possible to diagnose Behcet's disease and, in particular, to differentially diagnose between active Behcet's disease and inactive Behcet's disease. Moreover, when the CD206 is used in the diagnosis, the results of the diagnosis will not vary depending on symptoms of the disease such as whether it is accompanied by ocular lesions, and thus it is possible to reliably and accurately diagnose the disease and to diagnose the pathogenesis of active Behcet's disease regardless of the types of prescription medicines.

Moreover, still another aspect of the present invention provides a method for providing information for the diagnosis of Behcet's disease, comprising the steps of providing a sample to be analyzed, contacting an antibody specific to macrophage mannose receptor 1 (CD206) with the sample, and measuring the expression level of CD206.

The information providing method may further include, before the step of measuring the expression level of CD206, the step of staining the macrophage mannose receptor 1 of the sample.

The information providing method may further include the step of comparing the measured expression level of CD206 with the expression level measured in a normal subject.

The sample to be analyzed may be blood collected from a subject, in which the pathogenesis of active Behcet's disease is to be determined, or peripheral blood mononuclear cells (PBMCs) isolated from the blood.

For an effective analysis, the cells may be fixed by a method commonly used in the art to which the present invention pertains and used in the analysis.

The method of measuring the protein level using the antibody specific to macrophage mannose receptor 1 may be performed by a method of forming an antigen-antibody complex by contacting the antibody with a biological sample.

A specific analysis method for measuring the protein level may be any one selected from the group consisting of Western Blotting, ELISA, immunoassay, immunodiffusion, immunoelectrophoresis, immunostaining, immunoprecipitation, complement fixation assay, flow cytometry using fluorescence activated cell sorter (FACS), protein chip, and a combination thereof.

The method for measuring the protein level may preferably be performed by staining peripheral blood mononuclear cells collected from a subject for the analysis and measuring the protein level by flow cytometry using FACS.

In an embodiment of the present invention, it was found that it was possible to diagnose the pathogenesis of Behcet's disease and the progression of the disease by providing information obtained by measuring the frequency of expression of CD 206 on the surface of peripheral blood mononuclear cells in active Behcet's disease patients, which was significantly higher than that in normal subjects or inactive Behcet's disease patients. Moreover, according to this, the results of the diagnosis will not vary depending on symptoms of Behcet's disease patients and prescription medicines, and thus it is possible to effectively diagnose the active Behcet's disease.

Hereinafter, the following Examples are provided to more specifically illustrate the present invention, and the scope of the present invention is not limited by those Examples.

### Preparation Example

### Experiment Preparation and Analysis Method

### 1. Preparation of Experimental Groups and Characterization

The patient groups consisted of 13 patients with Behcet's disease who were treated at the Department of Dermatology, Yonsei University Hospital, Seoul, Korea. The control group consisted of 6 healthy volunteers (three women and three men; mean age, 29.6±3.5 years), the patient group with inactive Behcet's disease (hereinafter, referred to as an inactive group) consisted of eight (six women and two men; mean age, 48.4±15.0 years) and the patient group with active Behcet's disease (hereinafter, referred to as an active group) consisted of five (four women and one man; mean age, 30.0±8.6 years). According to the International Study Group for the diagnosis of Behcet's disease, the presence of any two of the following symptoms in addition to recurrent oral ulceration is diagnostic: genital ulceration, skin lesions, joint involvement, and ocular lesions. The active Behcet's disease patients had at least two of the Behcet's disease symptoms and the inactive Behcet's disease patients who received anti-inflammatory medication were well controlled with no symptomatic states. Two of eight inactive Behcet's disease patients were transferred from the patient group with active Behcet's disease.

Written informed consent was obtained from all participants prior to enrolling them into this study in accordance with the guidelines of the Declaration of Helsinki Principles.

Detailed clinical characteristics and therapeutic history of these patients are presented in the following Tables 1 and 2.

TABLE 1

| Disease | | | | | | | | | | | | | HLA- | | |
|---|---|---|---|---|---|---|---|---|---|---|---|---|---|---|---|
| Condition | Patient | Age | Sex | OU | GU | SL | GI | JI | NEUR | VAS | OL | Pathergy | B51 | ESR | CRP |
| Active | A | 28 | M | + | + | + | − | − | − | − | − | − | − | 13 | <0.01 |
| Group | B | 31 | F | + | − | − | − | − | − | − | + | − | + | 65 | <0.01 |
| | C | 29 | F | + | + | + | − | + | − | − | − | − | − | 41 | 2.4 |
| | D | 19 | F | + | + | + | − | + | − | − | − | − | − | 20 | <0.01 |
| | E | 43 | f | + | + | + | − | − | − | − | − | − | + | 50 | 2.33 |
| Inactive | A | 28 | M | −− | −− | −− | −− | −− | −− | −− | −− | −− | − | 2 | <1.00 |
| Group | B | 31 | F | −− | −− | −− | −− | −− | −− | −− | −− | −− | + | 10 | <1.00 |
| | F | 59 | F | −− | −− | −− | −− | −− | −− | −− | −− | −− | − | 10 | <1.00 |
| | G | 64 | F | −− | −− | −− | −− | −− | −− | −− | −− | −− | + | 17 | <1.00 |
| | H | 53 | F | −− | −− | −− | −− | −− | −− | −− | −− | −− | − | 19 | <1.00 |
| | I | 45 | M | −− | −− | −− | −− | −− | −− | −− | −− | −− | − | 14 | <1.00 |
| | J | 39 | F | −− | −− | −− | −− | −− | −− | −− | −− | −− | − | 18 | 1.54 |
| | K | 68 | F | −− | −− | −− | −− | −− | −− | −− | −− | −− | − | 2 | <1.00 |

In the above Table 1, M represents male, F represents female, OU represents oral ulcers, GU represents genital ulcers, SL represents skin lesions, GI represents gastrointestinal inflammation, JI represents joint involvement, NEUR represents neurological involvement, VAS represents vasculitis, and OL represents ocular lesions.

As shown in the above Table 1, all five active Behcet's disease patients had severe manifestations consisting of oral ulcers with genital ulcers and skin lesions during the course of the disease. One patient (patient B) did not show genital ulcers and skin lesions, but did have ocular lesions. In addition, two patients (patient C and patient D) had joint complications. However, gastrointestinal infection, neurological involvement and vasculitis were not observed during the study period.

The laboratory tests performed were skin hyperreactivity (pathergy test), genetic testing (HLA-B51 detection), erythrocyte sedimentation rate (ESR), and C-reactive protein (CRP) level.

Patients with active Behcet's disease showed an acute-phase response leading to a significantly raised ESR than inactive Behcet's disease (37.8±21.39 mm/h and 11.5±6.76 mm/h, p=0.007; respectively). Moreover, the serum level of CRP was varied from <0.01 mg/dL to 2.4 mg/dL in active Behcet's disease patients, and in inactive Behcet's disease patients was from <1.0 mg/dL to 1.54 mg/dL.

Genetic factor HLA-B51 was positively detected in both active and inactive Behcet's disease patient groups. Finally, a pathergy test was performed; none of the active Behcet's disease patients showed a positive result (Table 1). Therefore, it was confirmed once more that it was impossible to differentially diagnose between active patients and inactive patients through the pathergy test or genetic testing.

After blood sampling, the active Behcet's disease patients (patients A and B) started drug treatment. As shown in the following Table 2, patient A was treated with colchicine, prednisolone, and azathioprine; patient B was treated with colchicine, prednisolone, azathioprine, and cyclosporine; patients F, G and K were treated with colchicine; patients H and J were treated with colchicine and aspirin; and patient I was treated with prednisolone.

TABLE 2

| Disease | | | | | | |
|---|---|---|---|---|---|---|
| Condition | Patient | Colchicine | prednisolone | azathioprine | aspirin | cyclosporine |
| Inactive | A* | + | + | + | − | − |
| group | B* | + | + | + | − | + |
| | F | + | − | − | − | − |
| | G | + | − | − | − | − |
| | H | + | − | − | + | − |
| | I | − | + | − | − | − |
| | J | + | − | − | + | − |
| | K | + | − | − | − | − |

In the above Table 2, patients A* and B* were improved from active Behcet's disease patients A and B.

### 2. Cell Preparation

Whole blood was collected from the control group, the active group, and the inactive group, respectively, and cells were isolated and prepared for the analysis. Peripheral blood mononuclear cells (PBMCs) were isolated from heparinized venous blood by ACK lysing buffer. The isolated cells were washed twice in phosphate buffered saline (PBS) and then resuspended in PBS. The cell suspensions were finally adjusted to a concentration of 1×10⁶ cells/ml and were processed further for cellular staining studies.

### 3. Analysis

3.1 Flow Cytometry

The isolated PBMCs were surface-stained with anti-human antibodies for 30 minutes at 4° C. in the dark.

The anti-human antibodies used for each gene were anti-human CD11b FITC (Clone: ICRF44, eBiosciences, USA), anti-human CD14 PE-Cyanine 7 (Clone: 61D3, eBiosciences, USA), anti-human CD16PE (Clone: eBioCB16, eBiosciences, USA), anti-human CD32 APC (Clone: 6C4, eBiosciences, USA), and PE-Cy™5 mouse anti-human CD206 (Clone: 19.2, BD Biosciences Pharmingen, USA). In the surface staining, fluorescein isothiocyanate was used for CD11b, phycoerythrin was used for CD14 and CD16, allophycocyanin was used for CD32, and peridinin chlorophyll protein was used for CD206. Isotype control antibodies were used to estimate the non-specific binding of target primary antibodies. Stained cells were analyzed by flow cytometry using a FACS Canto II (Becton Dickinson, USA).

3.2 Enzyme-Linked Immunosorbent Assay (ELISA)

Serum was obtained from the patient groups and the healthy control group and analyzed using commercially available ELISA kits for the detection of IL-10 according to the manufacturer's instructions. ELISA values of each sample were measured at a wavelength of 450 nm using Bio-Rad 170-6850 microplate reader (Bio-Rad, USA). The mean and standard deviation were calculated using ELISA values determined for each well.

3.3 Transmission Electron Microscopy (TEM)

PBMCs were isolated from whole blood of the healthy control group, the inactive Behcet's disease patient group, and the active Behcet's disease patient group, and the morphological changes were observed using EM 902A transmission electron microscope (Zeiss, Germany).

To observe the cells using TEM, the isolated cells were fixed using Karnovsky's fixative solution (2% paraformaldehyde, 2% glutaraldehyde, 0.5% calcium chloride in cacodylate buffer, pH 7.2) for 30 minutes, washed with cacodylate buffer, dehydrated in a series of graded ethanol (from 20% to 100%), and embedded in Epon mixture. After polymerization in a 60° C. oven, ultrathin sections were cut using on Reichert Jung Ultracut S (Leica, Austria), stained with uranyl acetate and lead citrate, and analyzed by TEM.

3.4 Statistical Analysis

Statistical analysis was performed using SPSS 11.0 software (SPSS, USA) and analyzed by Kruskal-Wallis Test and Bonferroni correction. A value of p<0.05 was considered statistically significant.

### Experimental Example

### Characterization of Expression for Marker for Detection of Active Behcet's Disease Patients

### 1. Characterization of Surface Expression on PBMCs

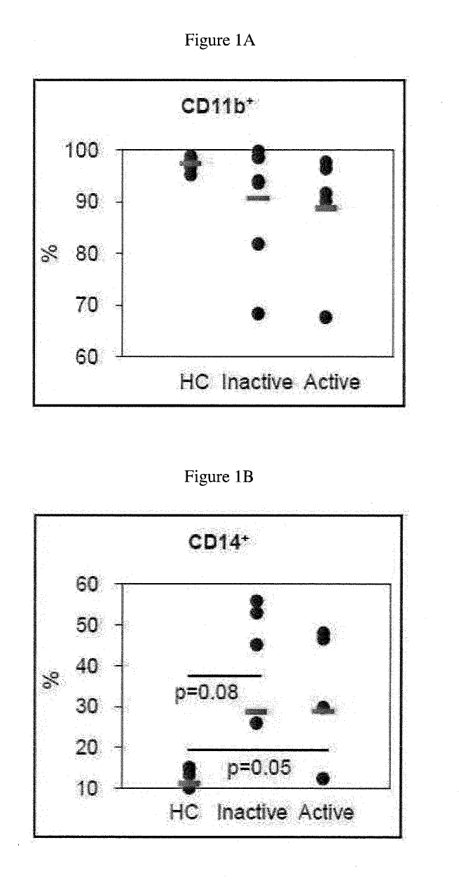

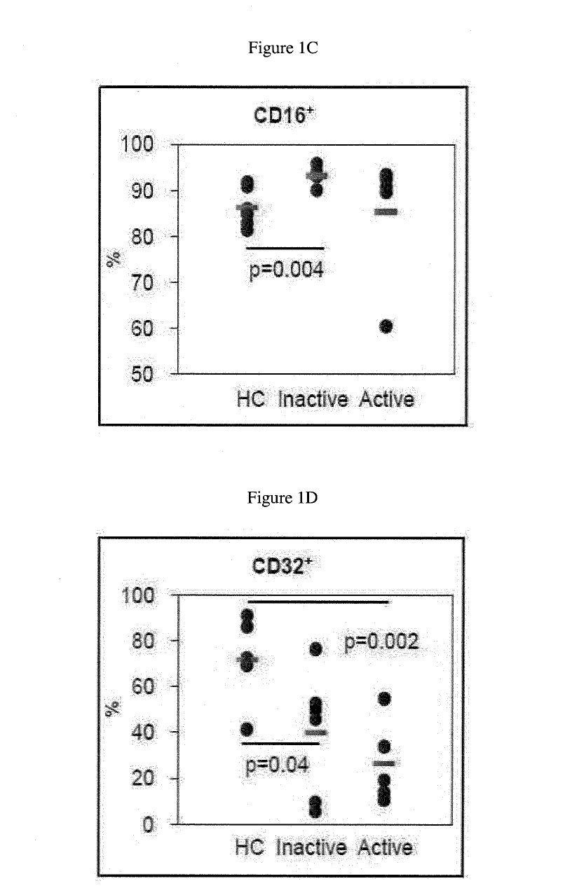

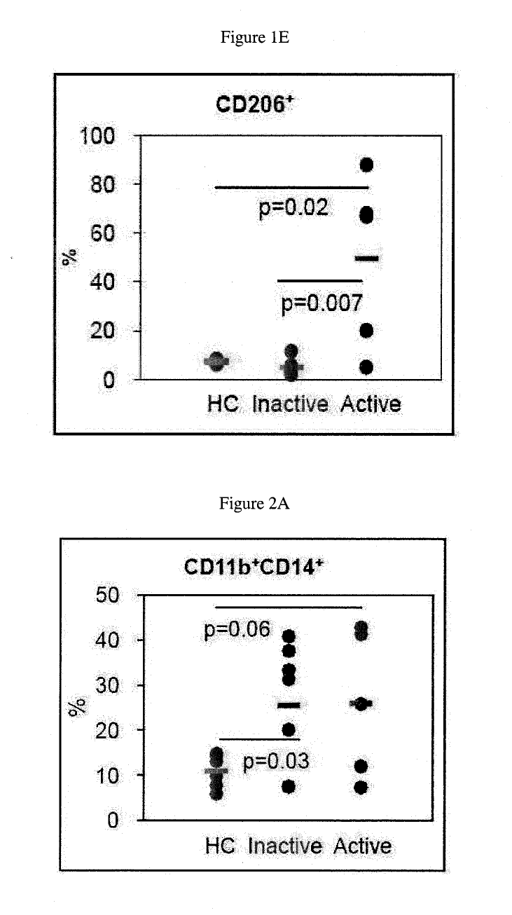

To identify differently expressed cell surface markers between active and inactive Behcet's disease patients, PBMCs were isolated from the healthy control group, the inactive Behcet's disease patient group, and the active Behcet's disease patient group, respectively, labeled with antibodies, and analyzed by flow cytometry. The results are shown in FIG. 1.

As shown in FIG. 1A, the CD11b⁺ monocyte marker was expressed 91.5±10.9% in the active Behcet's disease patient group (Active) and 88.8±12.2% in the inactive Behcet's disease patient group (Inactive), compared to 97.5±1.3% in the healthy control group (HC). Therefore, the CD11b⁺ monocyte marker was considered to be unsuitable as a marker for the diagnosis of active Behcet's disease as the differential expression between the patient groups and the control group and between the active group and the inactive group was not significant.

As shown in FIG. 1B, the monocyte marker CD14⁺ was expressed 28.9±18.7% (p=0.05) in the active Behcet's disease patient group and 30.8±21.4% (p=0.08) in the inactivate Behcet's disease patient group, compared to 11.1±3.7% in the control group.

As shown in FIG. 1C, CD16⁺ was expressed 93.9±2.4% in the inactive Behcet's disease patient group and 85.3±14% in the active Behcet's disease patient group.

As shown in FIG. 1D, CD32⁺ (FcγII) was expressed 46.6±30.3% (p=0.04) in the inactive Behcet's disease patient group and 71.7±17.4% (p=0.002) in the healthy control group, compared to 26.6±18.1% in the active Behcet's disease patient group.

As shown in FIG. 1E, CD206⁺ (mannose receptor marker) was expressed 7.4±0.8% (p=0.02) in the healthy control group and 4.7±3.1% (p=0.007) in the inactive Behcet's disease patient group, compared to 49.7±35.2% in the active Behcet's disease patient group patients. Therefore, it was confirmed that the surface expression on PBMCs in active Behcet's disease patients was higher than that without Behcet's disease and inactive Behcet's disease patients.

### 2. Characterization of CD11b⁺ Subsets

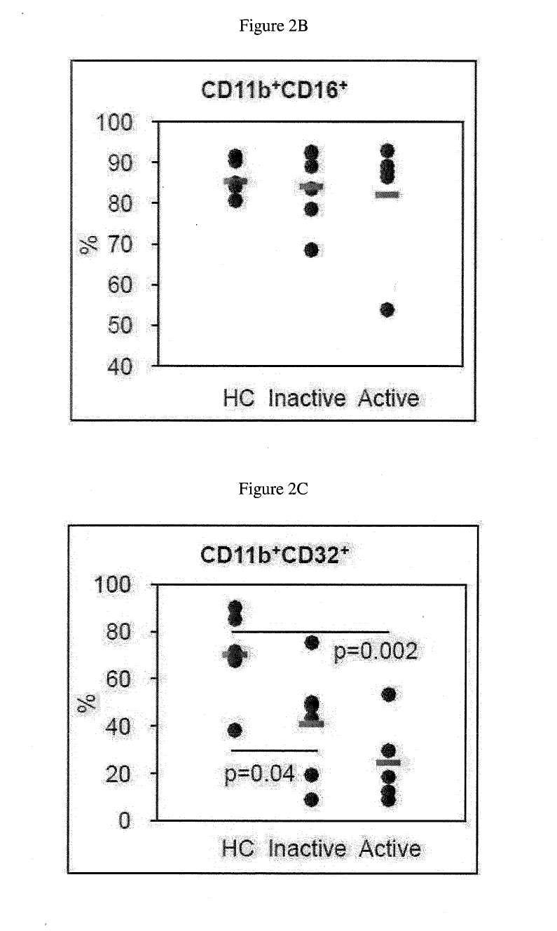

To determine the frequencies of co-expressed markers with CD11b⁺ and CD14⁺, CD16⁺ or CD32⁺, the frequencies of cells on which CD11b⁺CD14⁺, CD11b⁺CD16⁺ and CD11b⁺CD32⁺ were expressed were analyzed in PBMCs from the healthy control group, the active and inactive Behcet's disease patients by flow cytometry of section 3.1, and the results are shown in FIG. 2.

As shown in FIG. 2A, the frequencies of CD11b⁺CD14⁺ cells were 27.7±4.1% (p=0.03) in the inactive Behcet's disease patient group and 25.9±16.3% (p=0.06) in the active Behcet's disease patient group, compared to 11.0±3.7% in the control group. It was confirmed from these results that the frequencies of CD11b⁺CD14⁺ cells in the Behcet's disease patient groups were higher than those in the control group.

As shown in FIG. 2B, the frequencies of CD11b⁺CD16⁺ cells were 85.7±9.4% in the inactive Behcet's disease patient group and 82.2±15.9% in the active Behcet's disease patient group, compared to 85.5±4.7% in the control group. It was confirmed from these results that the frequencies of CD11b⁺CD16⁺ cells were not significantly different among the experimental groups.

As shown in FIG. 2C, the frequencies of CD11b⁺CD32⁺ cells were 24.8±18.0% (p=0.002) in the active Behcet's disease patient group and 47.8±27.8% (p=0.04) in the inactive Behcet's disease patient group, compared to 70.6±18.2% in the healthy control group. It was confirmed from these results that the frequencies of CD11b⁺CD32⁺ cells were significantly down-regulated in the Behcet's disease patient groups and the active Behcet's disease patients showed down-regulation of CD11b⁺CD32⁺ cells compared to the inactive Behcet's disease patients.

### 3. Characterization of CD14⁺CD16⁺ Subsets

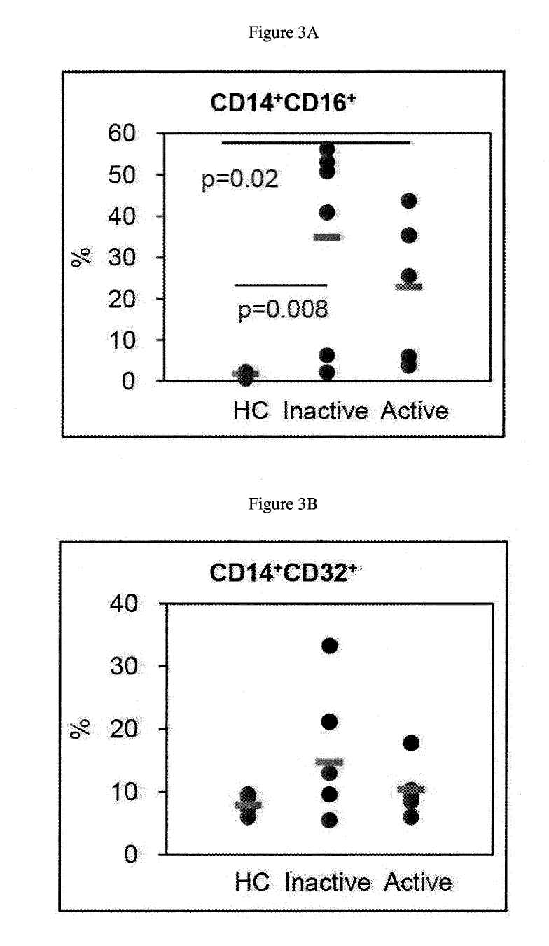

As shown in FIG. 1B, the frequencies of CD14+ cells were increased in both inactive and active groups as compared to those in the healthy control group. In this regard, to determine whether the expression of double positive cells was increased, the frequencies of CD14b⁺CD16⁺ and CD14b⁺CD32⁺ cells were analyzed by flow cytometry of section 3.1, and the results are shown in FIG. 3.

As shown in FIG. 3A, it was found that the frequencies of CD14⁺CD16⁺ cells were 36.1±22.4% (p=0.008) in the inactive patient group and 22.9±17.6% (p=0.02) in the active patient group, compared to 1.7±0.6% in the healthy control group

Moreover, as shown in FIG. 3B, the frequencies of CD14⁺CD32⁺ cells were 18.0±3.3% in the inactive Behcet's disease patient group, 10.4±4.4% in the active Behcet's disease patient group, and 7.9±1.3% in the healthy control group.

### 4. Determination of Increased Expression of Mannose Receptor CD206⁺

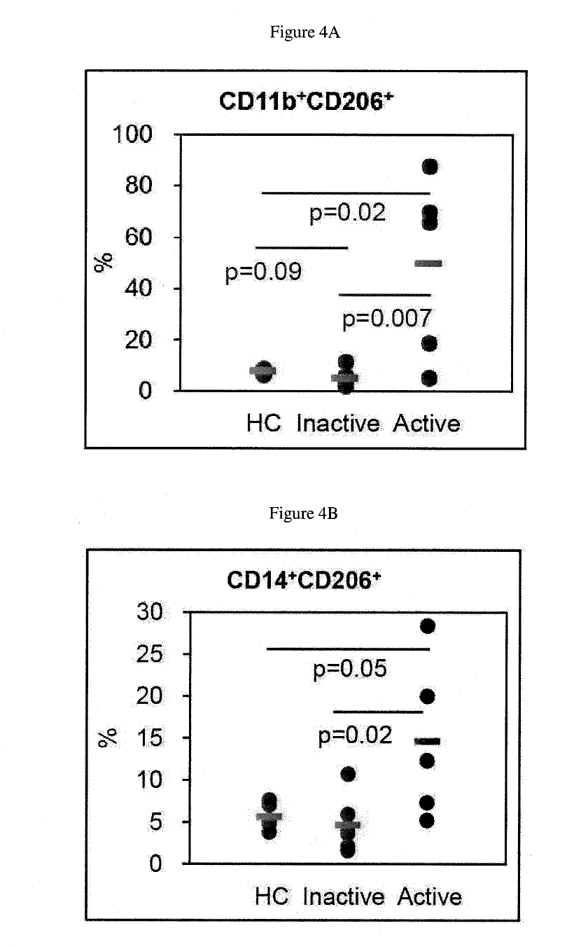

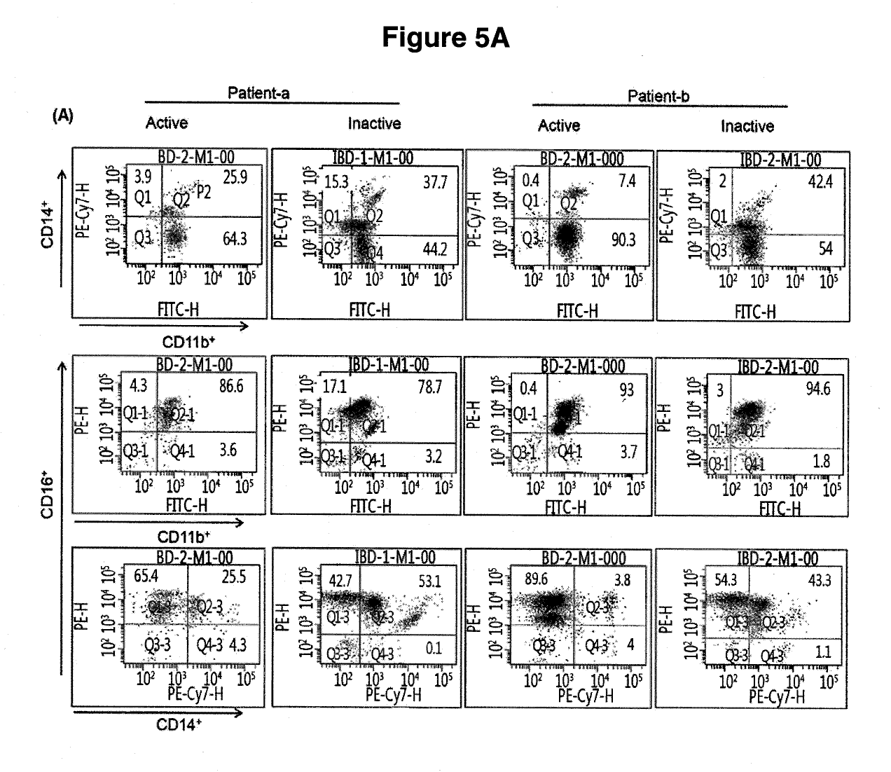

To determine the expression of mannose receptor in association with monocyte/macrophage subsets, the frequencies of CD11b⁺CD206⁺ and CD14⁺CD206⁺ cells in the control group and the active and inactive Behcet's disease groups were analyzed by flow cytometry of section 3.1, and the results are shown in FIG. 4

As shown in FIG. 4A, CD11b⁺CD206⁺ cells were expressed 7.4±0.9% in the control group and 4.4±3.1% (p=0.007) in the inactive Behcet's disease patient group, compared to 49.4±35.6%(p=0.05) in the active Behcet's disease patient group. Therefore, it was confirmed that the expression of CD11b⁺CD206⁺ cells in the active Behcet's disease patient group was significantly higher than that in other experimental groups

As shown in FIG. 4B, CD14⁺CD206⁺ cells were expressed 5.6±1.4% in the control group and 4.4±2.9% (p=0.02) in the inactive Behcet's disease patient group, compared to 14.6±9.6% in the active Behcet's disease patient group. Therefore, it was confirmed that the expression of CD14⁺CD206⁺ cells in the active Behcet's disease patient group was significantly higher than that in other experimental groups

Therefore, for both the single expression and the co-expression with other surface molecules, the expression of CD206⁺ cells was significantly high only in the active Behcet's disease patient group, and thus it was confirmed that the CD206⁺ cells could be used as a marker for the diagnosis of active Behcet's disease.

### 5. Determination of Change of Expression in Patients Improved from Active to Inactive

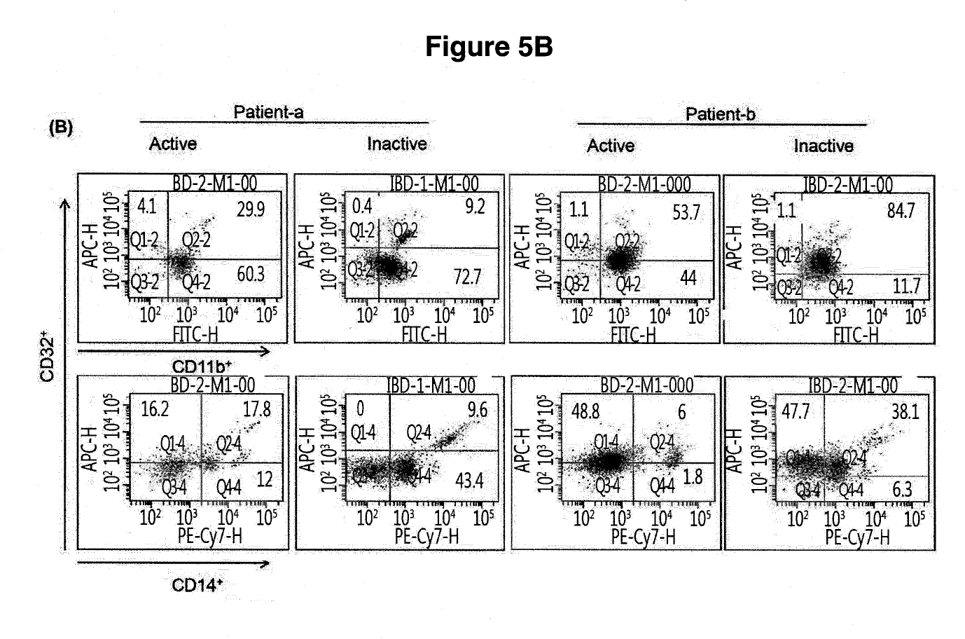

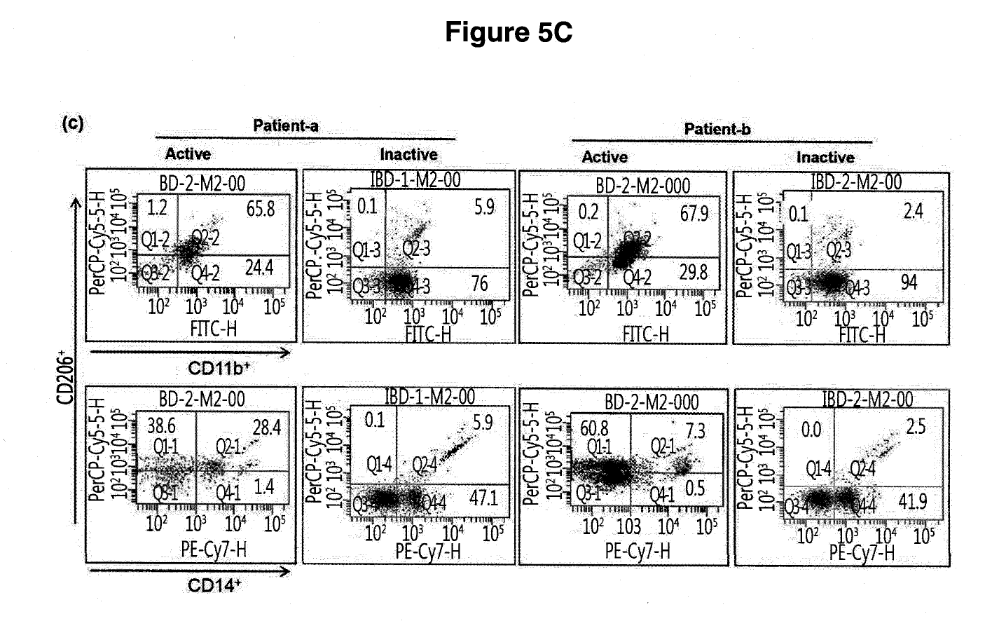

Among the five active Behcet's disease patients, two patients (patient A and B) were followed after treatment by the same manner as the above Preparation Example 1 (Table 2). When the symptoms of patients A and B changed from the active state to the inactive state, the surface expression of genes on PBMCs was analyzed by flow cytometry, and the results are shown in FIG. 5.

As shown in FIG. 5A, the frequency of CD14⁺ cells was increased from 29.8% (active stage) to 53.0% (inactive stage) in patient A and increased from 7.8% (active stage) to 44.4% (inactive stage) in patient B, and thus the frequency of CD14⁺ cells was increased in the inactive stage compared to the active state, which were the same tendency as those of FIG. 1B.

The frequency of CD11b⁺ cells was slightly decreased from 96.7% (active stage) to 96.4% (inactive stage) in patient A and from 90.2% (active stage) to 81.9% (inactive stage) in patient B. The frequency of CD14⁺CD11b⁺ cells was also increased to 37.7% in patient A and 42.4% in patient B at the inactive stage, compared to those (patient A: 25.9%, patient B: 7.4%) in the active stage (FIG. 5A). The frequency of CD16⁺ cells was slightly increased to 95.8% in patient A and 97.6% in patient B at the inactive stage, compared to those (patient A: 90.9%, patient B: 93.4%) in the active stage. The frequency of CD16′CD11b⁺ cells was lowered from 86.6% at the active state to 78.7% at the inactive stage in patient A, but was almost unchanged in patient B between 93.0% at the active stage and 94.6% at the inactive stage (FIG. 5A). Moreover, the frequency of double positive CD16⁺CD14⁺ cells was up-regulated from 25.5% in patient A and 3.8% in patient B at the active stage to 53.1% in patient A and 43.3% in patient B at the inactive stage (FIG. 5A)

As shown in FIG. 5B, the frequency of single CD32⁺ cells was decreased from 34.0% at the active stage to 9.6% at the inactive stage in patient A, but was increased from 54.8% at the active stage to 85.8% at the inactive stage in patient B. The frequency of double positive CD32⁺CD11b⁺ cells was decreased from 29.9% at the active stage to 9.2% at the inactive stage in patient A, but was increased from 53.7% at the active stage to 84.7% at the inactive stage in patient B. In addition, the frequency of CD32⁺CD14⁺ cells was decreased from 17.8% at the active stage to 9.6% at the inactive stage in patient A, but was increased from 6.0% at the active stage to 38.1% at the inactive stage in patient B. Therefore, the frequencies of CD32⁺, CD32⁺CD11b⁺, and CD32⁺CD14⁺ cells showed opposite pattern of result between patient A and B

As shown in FIG. 5C, the frequencies of CD206⁺ cells were highly up-regulated at the active stage in both patient A and B compared to those in the inactive stage. The frequencies of CD206⁺ cells at the inactive stage were 6.0% in patent A and 2.5% in patient B, but were 67% in patient A and 68.1% in patient B at the active stage. Furthermore, the frequencies of CD206⁺CD11b⁺ and CD206⁺CD14⁺ cells were 65.8% and 28.4%, respectively, in patient A and 67.9% and 7.3% respectively, in patient B at the active stage, but were 5.9% and 5.9%, respectively, in patient A and 2.4% and 2.5%, respectively, in patient B at the inactive stage, from which it was confirmed that the frequency of expression was significantly decreased at the inactive stage.

Summarizing the above results, the frequencies of CD32⁺ cells or double positive cells including CD32⁺ cells showed inconsistent results among the same experimental groups, and thus CD32⁺ cells cannot be used as a marker for the diagnosis of Behcet's disease. However, the frequencies of expression of CD206⁺ cells were high at the active stage without being affected by drugs prescribed to active patients, and thus CD206⁺ cells can be used as a useful marker for the diagnosis of active Behcet's disease.

### 6. Variations in Anti-Inflammatory Cytokine IL-10 after Improvement of Behcet's Disease

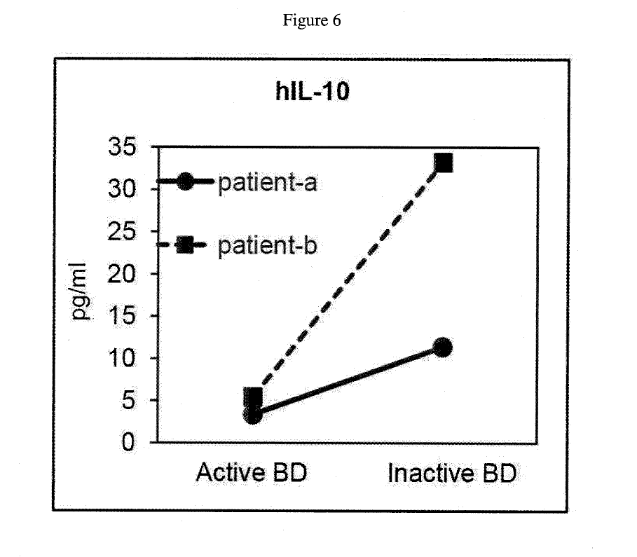

The anti-inflammatory cytokine IL-10 level was evaluated by ELISA as described in the above Preparation Example using serum collected from Behcet's disease patients A and B in their active and inactive stages, and the results are shown in FIG. 6.

As shown in FIG. 6, in patient B, the IL-10 level was 5.41 pg/ml at the active stage, but was significantly increased to 33.25 pg/ml at the inactive stage. In patient A, the IL-10 level was 3.36 pg/ml at the active stage, but slightly increased to 11.39 pg/ml at the inactive stage.

### 7. Observation of Morphology of Polymorphonuclear Neutrophils

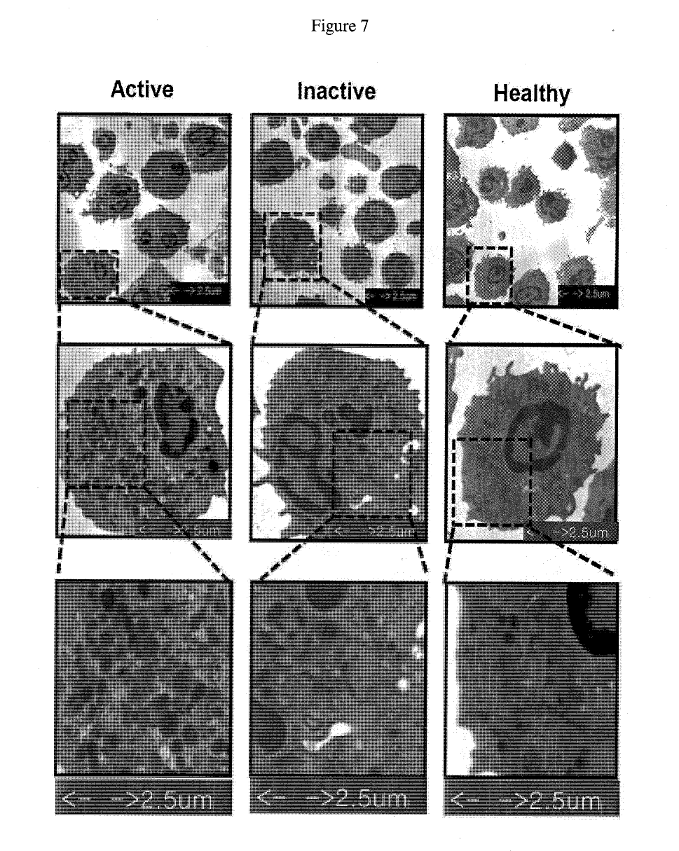

To observe the intracellular changes occurred at different stages of Behcet's disease, PBMCs were isolated from whole blood of the healthy control group, the inactive Behcet's disease patient group, and the active Behcet's disease patient group, and observed with TEM, and the results are shown in FIG. 7

As shown in FIG. 7, PBMCs had a normal structural appearance in the healthy control group and the inactive Behcet's disease patient group, but huge azurophilic granules were aggregated in the cytoplasm of the neutrophils in PBMCs of the active Behcet's disease patient group.

According to the composition for the diagnosis of Behcet's disease, the kit for the diagnosis of Behcet's disease, and the information providing method using the same in accordance with the present invention, it is possible to diagnose the pathogenesis of Behcet's disease without being affected by the symptoms of Behcet's disease or the prescription drugs and differentially diagnose between active Behcet's disease and inactive Behcet's disease.

While the invention has been shown and described with reference to certain preferred embodiments thereof, it will be understood by those skilled in the art that various changes in form and details may be made therein without departing from the spirit and scope of the invention as defined by the appended claims. Therefore, the scope of the invention is defined not by the detailed description of the invention but by the appended claims, and all differences within the scope will be construed as being included in the present invention.

What claimed is:

1. A composition for the diagnosis of Behcet's disease, comprising an agent for measuring the expression level of macrophage mannose receptor 1 (CD206).

2. The composition of claim 1, wherein the agent is an antibody specific to CD206.

3. The composition of claim 2, wherein the antibody is any one selected from a polyclonal antibody, a monoclonal antibody, a recombinant antibody, and a combination thereof.

4. A kit for the diagnosis of Behcet's disease, comprising an agent for measuring the expression level of macrophage mannose receptor 1 (CD206).

5. A method for providing information for the diagnosis of Behcet's disease, the method comprising the steps of:
providing a sample to be analyzed;
contacting an antibody specific to macrophage mannose receptor 1 (CD206) with the sample; and
measuring the expression level of CD206.

6. The method of claim 5, wherein the expression level of CD206 is measured by any one selected from the group consisting of Western Blotting, ELISA, immunoassay, immunodiffusion, immunoelectrophoresis, immunostaining, immunoprecipitation, complement fixation assay, flow cytometry, protein chip, and a combination thereof.

7. The method of claim 5, wherein the sample to be analyzed comprises peripheral blood mononuclear cells (PBMC) isolated from whole blood of a subject.
